(19) United States
(12) Patent Application Publication
(10) Pub. No.: US 2017/0205808 A1
(43) Pub. Date: Jul. 20, 2017

(54) METHOD FOR REVERSED MODELING OF A BIOMEDICAL MODEL

(71) Applicant: 3D Global Biotech Inc., Taipei Ciity (TW)

(72) Inventors: Keng-Liang Ou, Taipei City (TW); Yu-Hao Chan, Taipei City (TW); Han-Yi Cheng, Taipei City (TW)

(21) Appl. No.: 15/480,176

(22) Filed: Apr. 5, 2017

Related U.S. Application Data
(63) Continuation-in-part of application No. 14/304,962, filed on Jun. 15, 2014, now abandoned.

(51) Int. Cl. G05B 19/4099; B33Y 10/00; A61L 2/14; B33Y 50/02; B33Y 80/00; B29C 67/00; G06F 19/00; B33Y 40/00

(52) U.S. Cl. CPC G05B 19/4099; G06F 19/3437; B33Y 10/00; B33Y 40/00; B33Y 50/02; B33Y 80/00; B29C 67/0088; B29C 67/0081; A61L 2/14; G05B 2219/35134; G05B 2219/49007; B29L 2031/7532

(57) ABSTRACT

A method for reversed modeling of biomedical model of the present invention comprises at least the following steps: (a) Obtaining the data of a first model; (b) Transferring data of the first model into data of a second model which are applied to a rapid Prototyping machine; (c) Entering data of the second model into the rapid prototyping machine; (d) Laying model materials, coating adhesives and continuing sterilization by the rapid prototyping machine and stacking until a biomedical model is made; wherein sterilization is done during stacking medical model, therefore keeping the biomedical model sterile inside is achieved.

### FIELD OF THE INVENTION

This is a continuation-in-part application of applicant's U.S. patent application Ser. No. 14/304,962, filed on Jun. 15, 2014.

The present invention relates to a method for reversed modeling of a biomedical model, particularly to a method for reversed modeling of a biomedical model, wherein sterilization continues during printing of the biomedical model, so that the biomedical model is kept sterile.

### BACKGROUND OF THE INVENTION

Reversed engineering is different from conventional manufacturing method. It is a method, wherein a prototype is made before continual reproduction. Commonly there are analogical and digital reversed engineering.

For the conventional analogical reversed engineering e.g. coordinate milling is used for manufacturing proportional mold, however this way of manufacturing is difficult to modify.

Digital reversed engineering is digital measuring of prototype of the product, whereby data of digitized size are obtained. The digitized sizes are easy for reproduction and modification, thus the digital reversed engineering has gradually substituted the conventional reversed engineering. In manufacturing of digital reversed engineering there are several ways to use digitized size data of prototype for manufacturing, for example, manufacturing mold for production or using a rapid prototyping machine to manufacture end product directly.

Besides, in the medical field today implant, such as bone, is used for medical treatment. Since there are various kinds of implant, data of digitized size of the required implant can be obtained by means of CT or NMR, then entered into digital reversed engineering to manufacture implant through a rapid prototyping machine. Thence many adaption problems between implant and patient are avoided.

Since implant is to put into an organism, sterilization is very important. However, in the technique of rapid prototyping today, only surface sterilization can be done on the end product after a manufacturing process. So it is difficult to keep the product sterile inside.

In view of these disadvantages the inventor tried the continuous testing and improvement and developed the present invention.

### SUMMARY OF THE INVENTION

The main object of the present invention is to provide a method for reversed modeling of a biomedical model, wherein the biomedical model is kept sterile inside.

For achieving above object, the method for reversed modeling of a biomedical model of the present invention comprises the following steps: (a) Obtaining data of a first model; (b) Transferring the data of the first model by computer into data of a second model which match to rapid Prototyping machine; (c) Entering the data of the second model into a rapid prototyping machine; (d) Laying model materials, coating adhesives and continuing sterilization through the rapid prototyping machine and stacking until a biomedical model is made; wherein sterilization is done during stacking the medical model, therefore keeping a biomedical model sterile inside is achieved.

Preferably, the said data of the first model are obtained by means of CT, whereby a biomedical model of hard tissue is made.

Preferably, the said data of the first model are obtained by means of NMR, whereby a biomedical model of soft tissue is made.

Preferably, the step (b) includes modifying data of the first model according to predetermined requirement, to make a biomedical model.

Preferably, a low-temperature plasma is used in the action of a rapid prototyping machine for sterilization.

Other aspects and advantages of the present invention will become apparent from the following detailed description, taken in conjunction with the accompanying drawing, illustrating by way of example the principles of the present invention.

### BRIEF DESCRIPTION OF THE DRAWING

FIG. 1 is a flow chart of the method for reversed modeling of a biomedical model of the present invention.

FIG. 2 is a schematic view showing image data of a hard tissue of an organism obtained by a CT apparatus according to the present invention.

FIG. 3 is a schematic view showing image data of a soft tissue of an organism body obtained b a NMR apparatus according to the present invention.

FIG. 4 is a schematic view of a 3D simulation model built by the image data according to the present invention.

FIG. 5 is a schematic view showing modifying first model data according to the present invention.

FIG. 6 is a schematic view showing laying model materials by a rapid prototyping machine according to the present invention.

FIG. 7 is a schematic view showing laying another model materials by a rapid prototyping machine according to the present invention.

FIG. 8 is a schematic view showing coating adhesives by a rapid prototyping machine according to the present invention.

FIG. 9 is a schematic view showing a sterilization performed by a rapid prototyping machine according to the present invention.

FIG. 10 is a schematic view showing a low-temperature plasma sterilization method according to the present invention.

### DESCRIPTION OF THE PREFERRED EMBODIMENTS

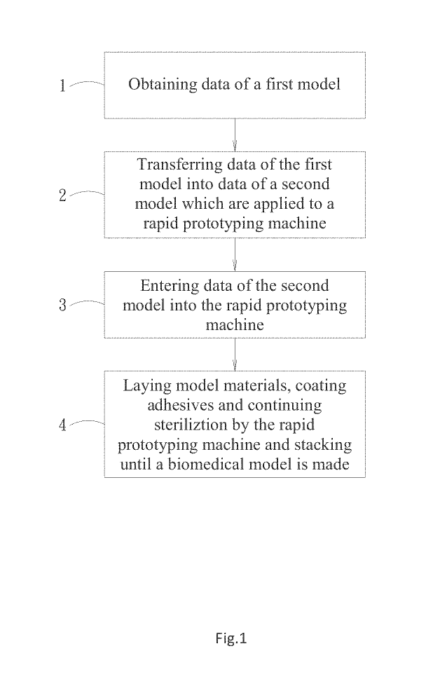

As shown in FIG. 1, the method for reversed modeling of a biomedical model of the present invention comprises at least the following steps: (a)1 Obtaining the data of a first model; (b)2 Transferring data of the first model by computer into data of a second model which are applied to a rapid prototyping machine; (c)3 Entering the data of the second model into a rapid prototyping machine; (d)4 Laying model materials, coating adhesives and continuing sterilization through the rapid prototyping machine and stacking until a biomedical model is made; wherein sterilization has is done during stacking the said biomedical model, therefore keeping the biomedical model sterile inside is achieved.

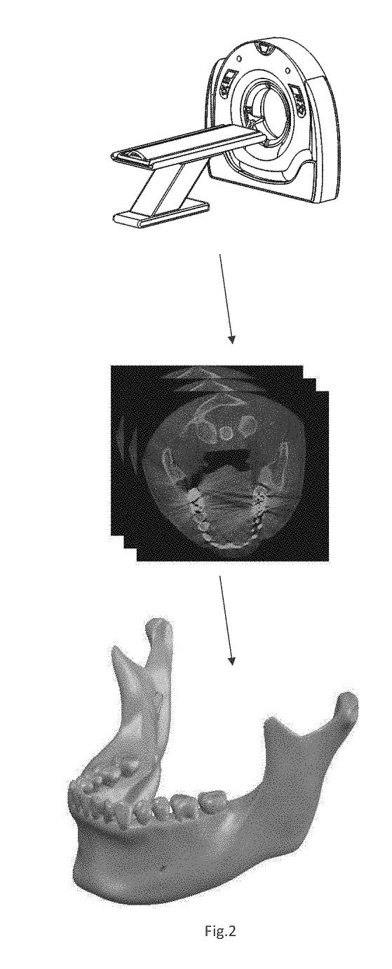

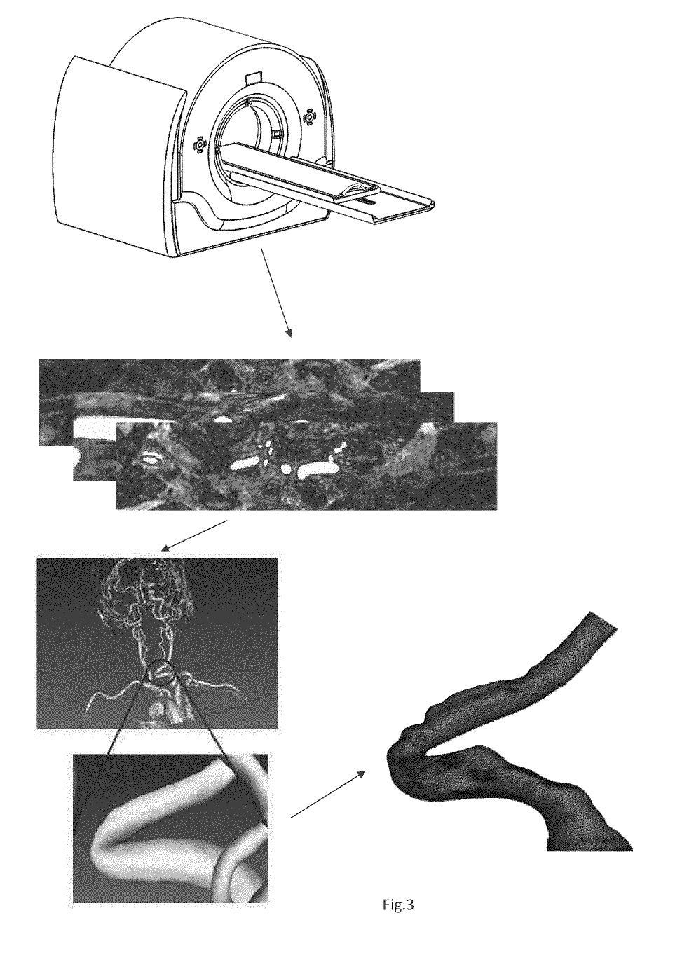

As shown in FIG. 2˜3, the step (a) is obtaining data of a first model, the data of the said first model are obtained by CT (as shown in FIG. 2) or NMR (as shown in FIG. 3), wherein the CT is used for obtaining image data of hard tissue of an organism, for a biomedical model of generating hard tissue, while the NMR is used for obtaining image data of soft tissue of an organism, for a biomedical model of generating soft tissue.

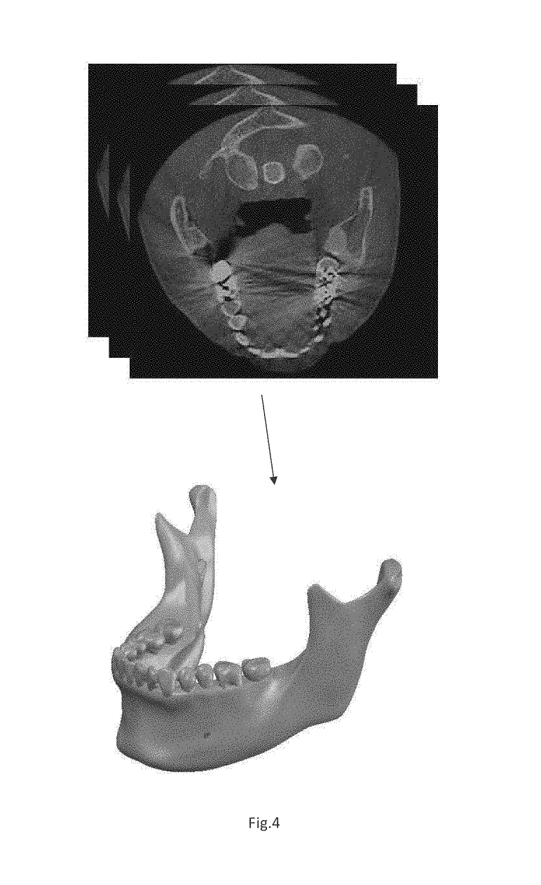

As shown in FIG. 4, the step (b) is transferring data of the first model by computer into data of a second model, which are applied to a rapid prototyping machine. Since data of the first model obtained by CT or NMR are sectional image data, which can not only easily build a 3D simulated model, but also can be transferred by computer into data of a second model, which are applied to a rapid prototyping machine.

As shown in FIG. 5, according to computer technique today, one can modify image data by computer, for example, the modify grayscale value, therefore a user can modify data of the first model by computer, to make a biomedical model according to predetermined requirement.

The step (c) is entering data of the second model into a rapid prototyping machine. A rapid prototyping machine is machine which builds a three dimensional model through layers of stacking. It is conventional art and hence will not be described here.

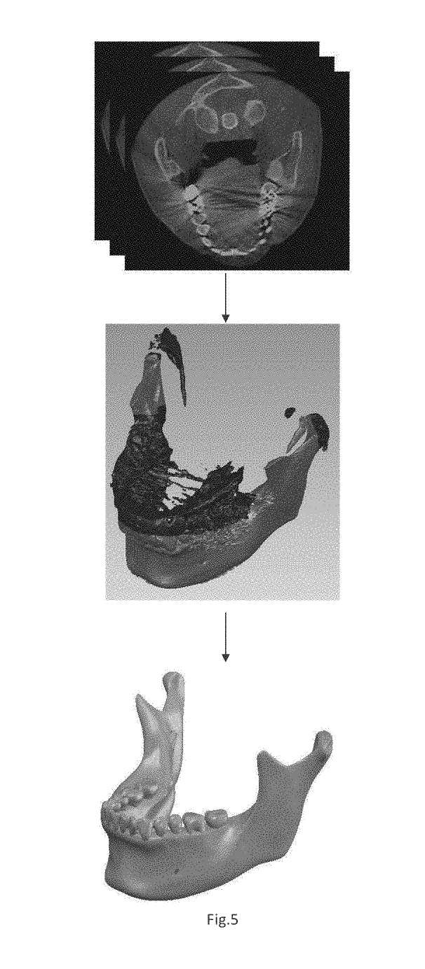

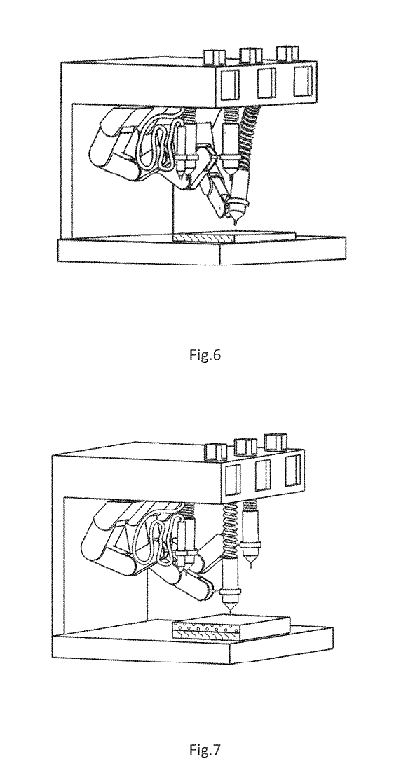

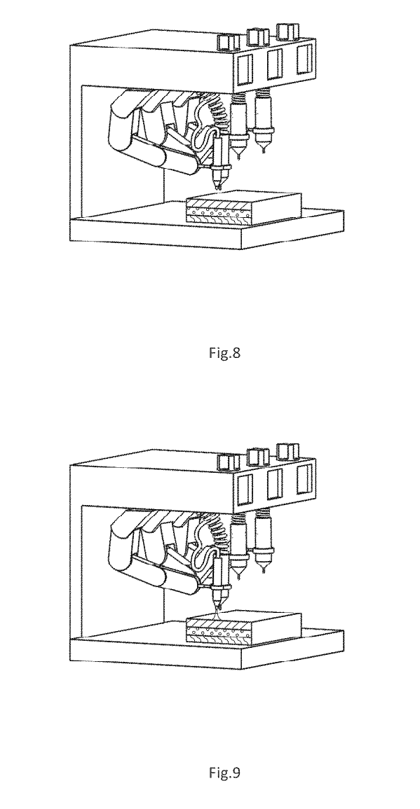

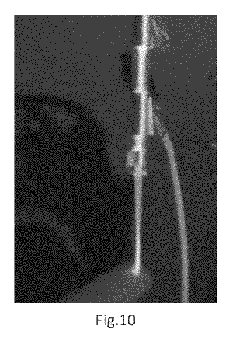

As shown in FIG. 6˜10, the step (d) laying model materials (as shown in FIG. 6˜7), coating adhesives (as shown in FIG. 8) and sterilizing (as shown in FIG. 9˜10) repeatedly through a rapid prototyping machine and stacking continually until a biomedical model is made. The action of a rapid prototyping machine will be described by preferred embodiments for demonstration.

As shown in FIG. 6˜7, action of a rapid prototyping machine for laying model materials is principally laying model materials according to data of the second model in a defined scope. Therefore, various powdery model materials are applicable, wherein biomedical model of macromolecule materials, like PVC (Polyvinyl Chloride), ABS (Acryonitrile-Butadiene-Styrene), PP (Polypropylone) and fluoropolymers, are preferred.

As shown in FIG. 8, action of a rapid prototyping machine for coating adhesives is coating adhesives according to data of the second model in a predetermined scope laid with model materials, so that the model materials cement together in the predetermined scope. For avoiding intolerance of the organism, biomedical hydrogel (proteinoid) is applied for using as adhesive.

As shown in FIG. 9˜10, action of a rapid prototyping machine for sterilization is taken at least in a predetermined scope coated with adhesives. Preferably low-temperature plasma is used for sterilization, wherein wave energy stimulates gas, so that ions and molecules collide with each other to produce radicals, thereby the metabolism of micro-organisms is destroyed. Advantages of this kind of sterilization are as following: that sterilization can be done under 50° C.; there are no toxic remnants in environment (Oxygen and water); cycle of sterilization is short and it is feasible to handling medical equipment of low heat resistance/low moisture resistance; due to the action for sterilization, keeping the biomedical model sterile inside is achieved.

While preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

1. A method for reversed modeling of a biomedical model comprises at least the following steps:
(a) Obtaining data of a first model;
(b) Transferring the data of the first model by computer into data of a second model which are applied to a rapid Prototyping machine;
(c) Entering data of the second model into a rapid prototyping machine;
(d) Laying model materials, coating adhesives and continuing sterilization through the rapid prototyping machine and stacking until a biomedical model is made.

2. The method for reversed modeling of a biomedical model of claim 1, wherein in the action of the rapid prototyping machine for sterilization plasma sterilization is used.

3. The method for reversed modeling of a biomedical model of claim 2, wherein in the action of the rapid prototyping machine for sterilization low-temperature plasma sterilization is used.

4. The method for reversed modeling of a biomedical model of claim 1, wherein data of the first model are obtained by means of CT.

5. The method for reversed modeling of a biomedical model of claim 4 wherein in the action of the rapid prototyping machine for sterilization plasma sterilization is used.

6. The method for reversed modeling of a biomedical model of claim 5, wherein in the action of the rapid prototyping machine for sterilization low-temperature plasma sterilization is used.

7. The method for reversed modeling of a biomedical model of claim 1, wherein data of the first model are obtained by means of NMR.

8. The method for reversed modeling of a biomedical model of claim 7, wherein in the action of the rapid prototyping machine for sterilization plasma sterilization is used.

9. The method for reversed modeling of a biomedical model of claim 8, wherein in the action of the rapid prototyping machine for sterilization low-temperature plasma sterilization is used.

10. The method for reversed modeling of a biomedical model of claim 1, wherein the step (b) includes modifying data of the first mode.

11. The method for reversed modeling of a biomedical model of claim 10, wherein in the action of the rapid prototyping machine for sterilization plasma sterilization is used.

12. The method for reversed modeling of a biomedical model of claim 11, wherein in the action of the rapid prototyping machine for sterilization low-temperature plasma sterilization is used.

13. The method for reversed modeling of a biomedical model of claim 10, wherein data of the first model are obtained by means of CT.

14. The method for reversed modeling of a biomedical model of claim 13, wherein in the action of the rapid prototyping machine for sterilization plasma sterilization is used.

15. The method for reversed modeling of a biomedical model of claim 14, wherein in the action of the rapid prototyping machine for sterilization low-temperature plasma sterilization is used.

16. The method for reversed modeling of a biomedical model of claim 10, wherein data of the first model are obtained by means of NMR.

17. The method for reversed modeling of a biomedical model of claim 16, wherein in the action of the rapid prototyping machine for sterilization plasma sterilization is used.

18. The method for reversed modeling of a biomedical model of claim 17, wherein in the action of the rapid prototyping machine for sterilization low-temperature plasma sterilization is used.
